(19) United States
(12) Patent Application Publication
(10) Pub. No.: US 2020/0390945 A1
(43) Pub. Date: Dec. 17, 2020

(54) MEDICAL CONTAINER

(71) Applicant: TELEFLEX LIFE SCIENCES UNLIMITED COMPANY, Hamilton (BM)

(72) Inventors: Ian Alexander BURTENSHAW, Oxford (GB); Kien Chung KWOK, Singapore (SG); Sylwia Ewa Krol POULSEN, Singapore (SG)

(21) Appl. No.: 16/762,925

(22) PCT Filed: Nov. 9, 2018
(86) PCT No.: PCT/EP2018/080801

§ 371 Date: May 9, 2020

(30) Foreign Application Priority Data
Nov. 10, 2017 (GB) 1718647.9

(51) Int. Cl. A61M 1/00; A61M 16/04; A61M 16/08

(52) U.S. Cl. CPC A61M 1/0019; A61M 16/0434; A61M 16/0816; A61M 16/0415

(57) ABSTRACT

Apparatus (2) for use with a laryngeal mask airway device (4), wherein the apparatus is configured to collect regurgitated fluid from a patient during use of the laryngeal mask airway device, wherein the apparatus is adapted for attachment to a gastric port (8) provided on the laryngeal mask airway device.

The present invention relates to apparatus for reducing the risk of contamination to medical personnel during use of an artificial airway device.

During medical emergencies, medical personnel often use artificial airway devices to establish an airway in an unconscious patient. Artificial airway devices such as the laryngeal mask airway device are well known devices that are useful for establishing airways in unconscious patients. In its most basic form a laryngeal mask airway device consists of an airway tube and a mask carried at one end of the airway tube, the mask having a peripheral formation often known as a “cuff” which is capable of conforming to and of fitting within, the actual and potential space behind the larynx of the patient so as to form a seal around the laryngeal inlet. The cuff can be inflatable, and in most variants it surrounds a hollow interior space or lumen of the mask, the at least one airway tube opening into the lumen. U.S. Pat. No. 4,509,514 is one of the many publications that describe laryngeal mask airway devices such as this. It is relatively easy to insert a laryngeal mask airway device into a patient and thereby establish an airway. Also, the laryngeal mask airway device is a “forgiving” device in that even if it is inserted improperly, it still tends to establish an airway. Accordingly, the laryngeal mask airway device is often thought of as a “life saving” device. Also, the laryngeal mask airway device may be inserted with only relatively minor manipulation of the patient's head, neck and jaw. Further, the laryngeal mask airway device provides ventilation of the patient's lungs without requiring contact with the sensitive inner lining of the trachea and the size of the airway established is typically significantly larger than the size of the airway established with an endotracheal tube. Also, the laryngeal mask airway device does not interfere with coughing to the same extent as endotracheal tubes. Largely due to these advantages, the laryngeal mask airway device has enjoyed increasing popularity in recent years.

One of the problems associated with the use of artificial airway devices is that regurgitated fluid may pass from the patient to the personnel operating the device. For example, in an emergency and/or ambulatory situation, the use of the artificial airway device may result in fluid from the stomach and/or blood from the patient passing through the artificial airway device to the face of the medical personnel and/or other people in proximity to the patient, thus increasing the risk of exposure to disease. There is therefore a need to develop apparatus that reduces the risk of contamination or spread of disease to the medical personnel and/or other people in the proximity of the patient and/or reduces the risk of contamination of the surroundings of the patient being treated.

It is an object of the present invention to seek to mitigate problems such as these.

According to a first aspect there is provided apparatus for use with a laryngeal mask airway device, wherein the apparatus is configured to collect regurgitated fluid from a patient during use of the laryngeal mask airway device, wherein the apparatus is adapted for attachment to a gastric port provided on the laryngeal mask airway device. Preferably, the apparatus is configured to collect regurgitated fluid such as blood and/or stomach fluid from the patient. Typically, the apparatus is configured to collect regurgitated fluid from a patient during insertion of the laryngeal mask airway device. Preferably, the apparatus is provided in the form of a fluid-tight container. Preferably, the container is a bag.

Advantageously, the apparatus of the present invention allows for regurgitated matter, blood and/or waste material from the patient to pass through the laryngeal mask airway device into the container, thus protecting medical personnel and/or others in proximity to the patient from exposure to regurgitated matter from the patient. Advantageously, the apparatus protects medical personnel and/or other people in proximity to the patient from exposure to potential diseases such as hepatitis, HIV, etc, and/or prevents contamination of the surroundings of the patient.

Preferably, the apparatus comprises means for attachment of the apparatus to the laryngeal mask airway device. Typically, the means for attachment comprises a connector to facilitate attachment of the apparatus to the laryngeal mask airway device. Preferably, the connector comprises silicone, polyvinylchloride or a synthetic rubber material. Typically, the connector is dimensioned such that it may be connected to a variety of sizes of gastric port provided on a laryngeal mask airway device. Preferably, the connector has an adjustable diameter. Advantageously, the adjustable diameter of the connector may facilitate the attachment of the connector to different sizes of gastric port.

Preferably, the apparatus comprises one or more vent adapted to allow pressure equalisation between the interior and exterior of the container. This can be important because regurgitation or vomiting can be sudden and forceful and breakage of the container or unexpected separation from the laryngeal mask is best avoided. In addition, if apparatus according to the invention is used with a laryngeal mask airway device with dual gastric lumens for suction, such as the LMA Protector™, suction can be applied to one lumen with the container still being in situ on the other lumen without suction being inhibited. Typically, the one or more vent is provided as one or more vent hole. Preferably, the one or more vent hole is provided on the connector. Preferably, the or each vent hole is adapted to prevent passage of liquid therethrough. For this purpose the or each vent hole may comprise a liquid impermeable membrane.

Preferably, the or each vent hole is provided with a releasable closure such as a cap.

Typically, the apparatus may comprise a plastics or metallic foil material. Preferably, the apparatus comprises a plastics foil or film. It is preferred that the apparatus is flexible and/or lightweight. Advantageously, this allows the apparatus to be stored in a compact manner when not in use. It is preferred that the apparatus is packaged such that it is sterile before use. Preferably, the apparatus is non-porous, advantageously allowing for isolation of the regurgitated material that has been collected. Typically, the apparatus is disposable. Advantageously, the apparatus is inexpensive to manufacture. Typically, the apparatus comprises polyethylene, polyvinylchloride or polypropylene.

According to a second aspect of the invention, there is provided a laryngeal mask airway device comprising apparatus in accordance with the first aspect of the invention. Advantageously, the laryngeal mask airway device of the second aspect allows for regurgitated matter, blood or other waste material from the patient to pass through the laryngeal mask airway device into the apparatus, thus protecting medical personnel and/or other people in proximity to the patient from exposure to regurgitated matter from the patient. Advantageously, the apparatus protects medical personnel and/or other people in proximity to the patient from exposure to potential diseases such as hepatitis, HIV, etc, and/or prevents contamination of the surroundings of the patient.

According to a third aspect, there is provided a kit comprising apparatus according to the first aspect and a laryngeal mask airway device.

According to a fourth aspect, there is provided a method of using apparatus according to the first aspect in combination with a laryngeal mask airway device, wherein the method involves the steps of attaching the apparatus of the first aspect to a laryngeal mask airway device, inserting the laryngeal mask airway device into the airway of a patient and collecting regurgitated fluid from the patient. Preferably, the apparatus is connected to the laryngeal mask airway device by means of a connector. Preferably, the connector is attached to a gastric port provided on the laryngeal mask airway device.

According to a fifth aspect, there is provided a method of manufacturing a laryngeal mask airway device according to the second aspect of the invention, comprising the steps of providing apparatus according to the first aspect of the invention and attaching the apparatus to a laryngeal mask airway device. It is preferred that the apparatus is attached to the laryngeal mask airway device by means of a connector. Preferably, the connector is provided on the apparatus. Preferably, the connector is attached to a gastric port provided on the laryngeal mask airway device. Preferably, the connector has an adjustable diameter.

The invention will be further described by way of example and with reference to the following drawings, in which,

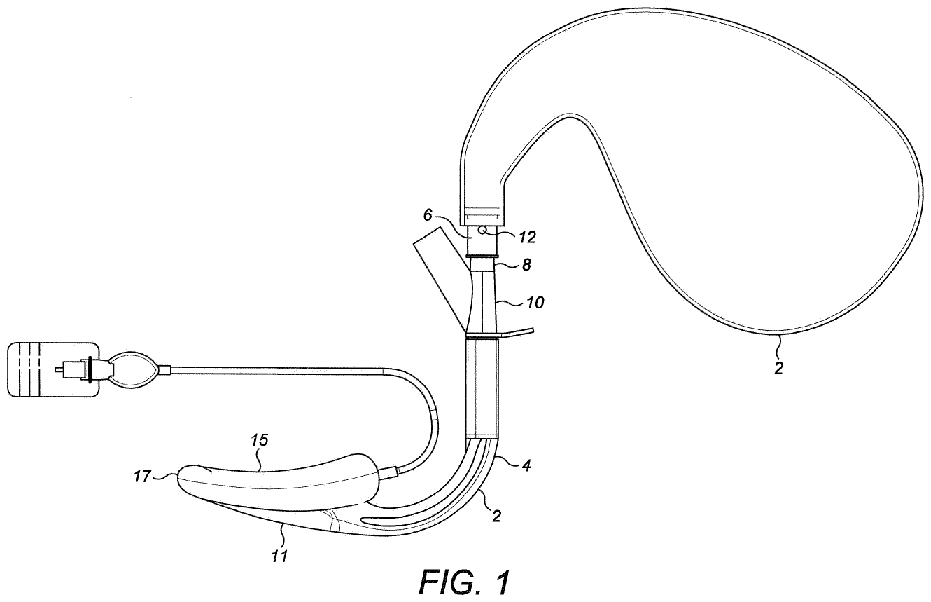

FIG. 1 is a side view of apparatus according to the invention in combination with a laryngeal mask airway device;

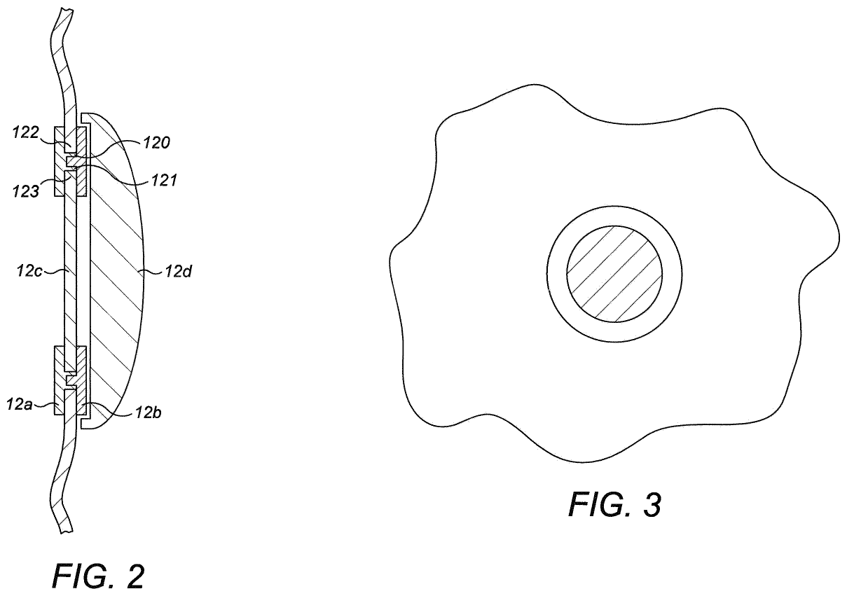

FIG. 2 is a side view of a vent hole for use with apparatus according to the invention; and

FIG. 3 is a front view of a vent hole for use with apparatus according to the invention.

With reference to FIG. 1, there is provided apparatus 2 for use with laryngeal mask airway device 4, wherein the apparatus 2 is configured to collect regurgitated fluid such as blood and/or stomach fluid from a patient during use (e.g. during insertion) of the laryngeal mask airway device. The apparatus is provided in the form of a container 2 which advantageously allows for regurgitated matter (such as stomach contents and/or blood) from the patient to pass from the laryngeal mask airway device into the container, thus protecting medical personnel and/or other people in proximity to the patient from exposure to regurgitated matter from the patient. Advantageously, the apparatus protects medical personnel and/or other people in proximity to the patient from exposure to potential diseases such as hepatitis, HIV, etc, and/or prevents contamination of the surroundings of the patient.

The apparatus 2 comprises a connector 6 to facilitate attachment of the apparatus 2 to the laryngeal mask airway device 4. The apparatus is attached to a gastric port 8 provided at the proximal end of the artificial airway device. The apparatus may be attached to a gastric tube 10 of the laryngeal mask airway device. Advantageously, the connector 6 may comprise an adjustable diameter to facilitate attachment of the connector 6 to the gastric port 8. The connector 6 may comprise silicone, polyvinylchloride or a synthetic rubber material.

The apparatus 2 comprises one or more vents 12, provided in the form of vent holes, to allow air to be evacuated from the apparatus. In the embodiment shown in FIG. 1, the one or more vent holes 12 are provided on the connector 6 as simple small diameter orifices. It is preferred however that the vents are liquid-tight to prevent escape of contained liquids. For this purpose each vent hole 12 is provided with a membrane that allows air to pass, but substantially prevents passage of liquid. An example of a suitable membrane material is PTFE.

Referring now to FIGS. 2 and 3 there is illustrated an alternative form of vent hole 12 for use with apparatus according to the invention. This simple structure can be easily incorporated into the wall of the container and consists of an inner mounting ring 12a, an outer mounting ring 12b, a membrane 12c and a cap 12d. Inner and outer mounting rings 12a,12b include mutually interengageable male and female flange parts 120, 121. When these are engaged the mounting rings together provide inner and outer mounting grooves 122, 123 dimensioned to securely trap the wall material of the container and the membrane. Cap 12d is removably attached by push-fitting.

Typically, the apparatus 2 may comprise a plastics or metallic foil material. Preferably, the apparatus 2 comprises a plastics film. Advantageously, the apparatus is inexpensive to manufacture and is typically disposable. Preferably, the apparatus comprises a material that is flexible and lightweight such that the apparatus may be stored in a compact form when it is not in use. Preferably, the apparatus is non-porous to allow for isolation of the regurgitated material. Preferably, the apparatus comprises polyethylene, polyvinylchloride and/or polypropylene.

With reference to FIG. 1, there is also provided a laryngeal mask airway device 4 comprising apparatus 2 configured to collect regurgitated fluid such as blood and/or stomach fluid from a patient during use (e.g. during insertion) of the laryngeal mask airway device. Typically, the laryngeal mask airway device 4 comprises silicone, silicone rubber or PVC. Advantageously, the apparatus may be used with different sizes of laryngeal mask airway devices. The laryngeal mask airway device comprises one or more gastric ports 8 for connection of the apparatus to the device. The apparatus is connected to the one or more gastric ports by means of a connector 6. The gastric port 8 is provided at the proximal end of the laryngeal mask airway device.

The laryngeal mask airway device 4 further comprises an airway tube 9, a backplate 11 attached to an airway tube 9, a peripheral inflatable cuff 15, and an outlet 17. The backplate 11 is attached to the airway tube 9. The device provides for gaseous communication between the airway tube 9 and the outlet 17.

In use, the apparatus 2 is connected to a gastric port 8 provided at the proximal end of the laryngeal mask airway device via connector 6. The connector 6 comprises one or more vent holes 12 to allow for air to be removed from the apparatus during use. The laryngeal mask airway device 4 is inserted into a patient in the usual manner associated with devices of this type. Regurgitated fluid from the patient is passed through the laryngeal mask airway device 4 and into container 2. Advantageously, this reduces the risk of medical personnel and/or other people in proximity to the patient being exposed to potential contamination from the patient, thus reducing the possible spread of disease.

With reference to FIG. 1, there is provided a kit comprising apparatus 2 and a laryngeal mask airway device 4, wherein the apparatus 2 is configured to collect regurgitated fluid such as blood and/or stomach fluid from a patient during use (e.g. during insertion) of the laryngeal mask airway device.

With reference to FIG. 1, there is also provided a method of of using apparatus 2 in combination with a laryngeal mask airway device 4, wherein the method involves the steps of attaching the apparatus 2 to the laryngeal mask airway device 4, inserting the laryngeal mask airway device into the airway of a patient and collecting regurgitated fluid from the patient. Preferably, the apparatus is connected to the laryngeal mask airway device by means of a connector 6. Preferably, the connector 6 is attached to a gastric port 8 provided on the laryngeal mask airway device.

With reference to FIG. 1, there is also provided a method of manufacturing a laryngeal mask airway device 4 comprising apparatus 2, comprising the steps of providing apparatus 2 and attaching the apparatus 2 to a laryngeal mask airway device 4. It is preferred that the apparatus 2 is attached to the laryngeal mask airway device 4 by means of a connector 6 provided on the apparatus. Preferably, the connector 6 is attached to a gastric port 8 provided on the laryngeal mask airway device.

1. An apparatus for use with a laryngeal mask airway device, wherein the apparatus is configured to collect regurgitated fluid from a patient during use of the laryngeal mask airway device, wherein the apparatus is adapted for attachment to a gastric port provided on the laryngeal mask airway device.

2. The apparatus according to claim 1, wherein the apparatus is configured to collect regurgitated fluid from the patient during insertion of the laryngeal mask airway device.

3. The apparatus according to claim 1, wherein the apparatus is provided in the form of a fluid-tight container.

4. The apparatus according to claim 3, wherein the container is a bag.

5. The apparatus according to claim 1, wherein the apparatus comprises means for attachment of the apparatus to the laryngeal mask airway device.

6. The apparatus according to claim 5, wherein the means for attachment comprises a connector to facilitate attachment of the apparatus to the laryngeal mask airway device.

7. The apparatus according to claim 6, wherein the connector has an adjustable diameter.

8. The apparatus according to claim 6, wherein the connector comprises silicone, polyvinylchloride or synthetic rubber.

9. The apparatus according to claim 3, wherein the apparatus comprises one or more vents adapted to allow pressure equalization between the interior and exterior of the container.

10. The apparatus according to claim 9, wherein the one or more vents is provided as one or more vent holes.

11. The apparatus according to claim 10, wherein the one or more vent holes are provided on the connector.

12. The apparatus according to claim 10, wherein at least one of the one or more vent holes is adapted to prevent passage of liquid therethrough.

13. The apparatus according to claim 10, wherein at least one of the one or more vent holes comprises a liquid impermeable membrane.

14. The apparatus according to claim 10, wherein at least one of the one or more vent holes is provided with a releasable closure such as a cap.

15. The apparatus according to claim 1, wherein the apparatus comprises a plastics or metallic foil material.

16. The apparatus according to claim 1, wherein the apparatus comprises a plastics or metallic foil material.

17. The apparatus according to claim 15, wherein the apparatus is flexible and/or lightweight.

18. The apparatus according to claim 15, wherein the apparatus is non-porous.

19. The apparatus according to claim 15, wherein the apparatus comprises polyethylene, polyvinylchloride or polyethylene.

20. A laryngeal mask airway device comprising the apparatus in accordance with claim 1.

21. A kit comprising a laryngeal mask airway device according to claim 20.

22. A method of using the apparatus according to claim 1 in combination with a laryngeal mask airway device, wherein the method comprises attaching the apparatus to the laryngeal mask airway device, inserting the laryngeal mask airway device into the airway of the patient, and collecting regurgitated fluid from the patient.

23. The method according to claim 22, wherein the apparatus is connected to the laryngeal mask airway device by means of a connector.

24. The method according to claim 22, wherein the connector is attached to a gastric port provided on the laryngeal mask airway device.

25. A method of manufacturing a laryngeal mask airway device, comprising providing the apparatus according to claim 1 and attaching the apparatus to the laryngeal mask airway device.

26. The method according to claim 25, wherein the apparatus is attached to the laryngeal mask airway device by a connector provided on the apparatus.

27. The method according to claim 25, wherein the connector is attached to a gastric port provided on the laryngeal mask airway device.
